(19) United States
(12) Patent Application Publication
(10) Pub. No.: US 2009/0060845 A1
(43) Pub. Date: Mar. 5, 2009

(54) DIAGNOSIS SUBSTANCE FOR APPLICATION IN A METHOD TO DIAGNOSIS PATHOLOGICAL TISSUE AND A METHOD FOR PRODUCTION OF SUCH A DIAGNOSIS SUBSTANCE

(76) Inventors: Jens Fehre, Hausen (DE); Ralf Nanke, Neunkirchen am Brand (DE); Martin Stetter, Muenchen (DE)

Correspondence Address: SCHIFF HARDIN, LLP;PATENT DEPARTMENT, 6600 SEARS TOWER, CHICAGO, IL, 60606-6473, US

(21) Appl. No.: 12/203,268

(22) Filed: Sep. 3, 2008

(30) Foreign Application Priority Data
Sep. 3, 2007 (DE) 10 2007 041 835.5

(51) Int. Cl. A61K 49/06; A61K 49/04

(52) U.S. Cl. 424/9.32; 424/9.4

(57) ABSTRACT

A diagnosis substance for application in a method for diagnosis of pathological tissue, contains at least one virus population with virus particles specifically binding to target molecules typical of a specific pathological tissue, with a label that is detectable with the use of a detection device being bound to the virus particles. In a method for production of such a diagnosis substance, the principle of directed biological evolution is applied.

### BACKGROUND OF THE INVENTION

1. Field of the Invention

The invention concerns a diagnosis substance for application in a method for diagnosis of pathological tissue and a method for production of such a diagnosis substance.

2. Description of the Prior Art

In the diagnosis of complex illnesses, differentiation between body tissues with different illnesses, and differentiation between different stages of a pathological tissue, are of decisive importance for the determining an optimal procedure for therapy. It is known to supply a diagnosis substance containing biomarkers to the tissue in question (usually via the blood stream) to detect a specific tissue type. The biomarkers contain a coupling molecule that specifically binds to a molecule (which here is designated as a target molecule) typical of a specific pathological tissue or a specific stage of such a tissue. Binding events at the tissue can be recognized by means of a label (for example a fluorescing dye molecule) detectable with a detection device and linked with the coupling molecule. A “target molecule” means not only individual molecules but also molecular structures that, for instance, have binding locations for a coupling molecule, that are formed from multiple molecules. In addition to biomarkers, the diagnosis substance can contain further components, for instance an aqueous solvent and additives. For example, in the case of the early hgPIN stage (high grade prostatic intraepithelial neoplasia) of prostate cancer, CEACAM-1 molecules (carcinoembryonic antigen-related cell adhesion molecules) are formed in the endothelium of the blood vessels directly adjacent to the cancer tissue. These molecules are thus indicators for the cited cancer stage. The goal of the diagnostics is to reliably establish the presence of such “indicator molecules” or target molecules. A biomarker with which this is possible must tether to the target molecule or the target structure with high selectivity and specificity, meaning that it must be able to “recognize” a specific binding partner from the entire set of possible binding partners with high probability and to reliably bond thereto. Moreover, the biomarker must remain bound at least until the aforementioned detection has concluded. With regard to the detection, it must be ensured that this can be implemented with an (optimally non-invasive) appropriate method.

Complex pathologies such as cancer, among other things, exhibit many characteristics, degrees of maturity and aggressiveness grades in addition to the stages mentioned above. For example, a separate molecular mechanism exists for the metastatic activity of each type of primary tumor (prostate, lung, intestine etc.) as well as given primary tumors for each target tissue of a metastasis. A large number of different biomarkers is required in order to be able to make a differential diagnosis in this context, which is in turn necessary for the selection of an optimal therapy. Methods similar to as in active substance development (drug discovery) are employed for their production. Such a method is, for example, “high throughput screening”, in which many thousands of samples of targets (thus of target molecules) are brought together with thousands of different molecule types in an automated manner. Molecules that bind well are thereby considered as potential biomarkers, but these must then still be optimized with regard to their binding properties, for example in different environmental conditions, as well as with regard to their biocompatibility and their ability to bind to a detectable label. The production of biomarkers according to this method is therefore relatively laborious and complicated.

### SUMMARY OF THE INVENTION

An object of the present invention is to provide a diagnosis substance that can be produced more simply, as well as to provide a corresponding production method.

The first cited object is achieved by a diagnosis substance according to the invention that contains at least one virus population with specific virus particles binding to target molecules typical of a specific pathological tissue, with a label that is detectable with the use of a detection device being bound to the virus particles. Naturally, only those viruses that are harmless to humans are used. Virus particles or virions have a coating composed mostly of different proteins, these proteins being genetically, variably encoded in the DNA, meaning that the virus can mutate the structure of the proteins, for instance to adapt to different host cells. Due to the genetic variability of the virus coating, the principle of reproduction and selection (thus a directed biological evolution) can be utilized in order to reconfigure a protein of the virus coating so that it specifically binds to the target molecule. M13 phages (completely harmless to humans) that proliferate with E. coli as host cells are particularly advantageous. These are thread-shaped phages with a thickness of approximately 6 nm and a length of up to 1 μm. They contain a single DNA ring that is surrounded by a casing made of a coat protein (here designated with Type 1). Additional proteins that are designated here with Type 2 and Type 3 are located at the ends of the phage. The cited proteins can easily be adapted to the most different binding partners via directed evolution, as is explained in detail further below.

### BRIEF DESCRIPTION OF THE DRAWINGS

FIG. 1 illustrates a virus particle bound to a target molecule, with a microbubble as a label.

FIG. 2 illustrates the binding of a virus particle to a persistent endothelial target molecule.

FIG. 3 is a flow chart of an embodiment of a production method in accordance with the invention for a specifically binding virus population.

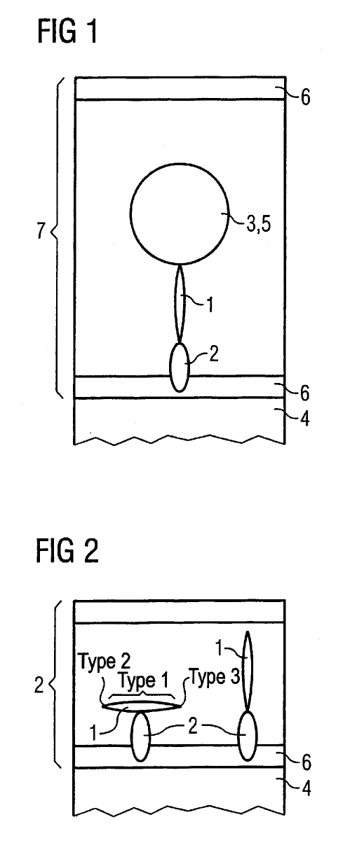

### DESCRIPTION OF THE PREFERRED EMBODIMENTS

After a diagnosis substance has been supplied (for instance via oral, intravenous or rectal administration) to a pathological tissue (referred to in the following as a prostate cancer) via the bloodstream, a specific binding occurs between a target molecule and a coat protein of a virus particle serving as a biomarker which, in a particularly preferred embodiment variant, is an M13 phage 1 that is exemplarily referenced in the following. M13 phages are a widespread tool in genetics, such that their use is advantageous insofar as known and reliable techniques can be used (for example in their reproduction). The thread-shaped M13 phage 1 has an essentially cylindrical coating extending nearly over its entire length, made from a single type of protein surrounding a single-stranded DNA ring (not shown). Additional proteins of Types 2 and 3 (respectively indicated in FIG. 2) are present at the ends of the phage 1. Both the terminal proteins of Types 2 and 3 and the protein of Type 1 of the phage coating are suitable are suitable for tethering to a target molecule 2 as well as to a label 3 detectable by a detection device. The label 3 can either be a molecule or a particle. The label is advantageously designed such that it is suitable for detection with the use of an imaging method. For example, it is possible to use ferromagnetic particles that are detectable with the use of MRT methods (MRT=magnetic resonance tomography) as a label 3. Such particles are naturally also detectable with the use of computed tomography (CT), but do not need to be ferromagnetic for this purpose. Here it is sufficient for the particles to be composed of a metal that is impermeable or only slightly permeable to x-rays, for example, an arbitrary metal. An accumulate of phages 1 in the cancer tissue 4 can also be detected when a dye that absorbs electromagnetic waves (for instance in the near infrared range) is used as a label 3. A dye fluorescing in the cited wavelength range is also advantageously conceivable. Electromagnetic radiation in the near infrared range has the property that it penetrates the body tissue relatively unhindered, such that it is detectable with probes (for instance with a rectal probe in the case of prostate cancer) set at a distance from the cancer tissue. An additional possibility to make an accumulation of phages 1 in the cancer tissue 4 visible is to use what are known as microbubbles 5 as labels 3. Microbubbles are tiny bubbles whose envelope is formed from a lipid double membrane, for example. The detection ensues via exposure with ultrasound and detection with corresponding ultrasound sensors.

Depending on the pathology in question, different indicator molecules or target molecules 3 act as binding partners for the phages 1. Given the treatment of prostate cancer, in which the present invention is particularly advantageously applicable, different molecule types form in the endothelium of the vascular wall 6 of blood vessels 2 of the cancer tissue 4 or the blood vessels 2 directly adjacent to the cancer tissue for respective different stages of the prostate cancer; for example, molecules known as CEACAM-1 molecules form in the case of the hgPIN stage, thus high-grade prostate neoplasy. In this case the phage 1 is bred (cultured) by the method explained in detail further below so that its coat protein selectively and specifically binds to CEACAM-1. A later cancer stage already located in the angiogenesis stage can be established using the growth factor VEGF or using the alpha(V)-beta(3)-integrins that are likewise formed, for example. The phages 1 used for differential diagnosis of the cited cancer stage are likewise adapted via a directed evolution method so that they selectively and specifically bind to the cited molecules. An administered diagnosis substance can now contain not only one type of a specific phage 1 but rather multiple different phages 1 binding to different target molecules. For example, it is thus advantageous when the diagnosis substance contains both phages 1 binding to CEACAM-1 and phages 1 binding to VEGF and/or alpha(V)-beta(3)-integrins.

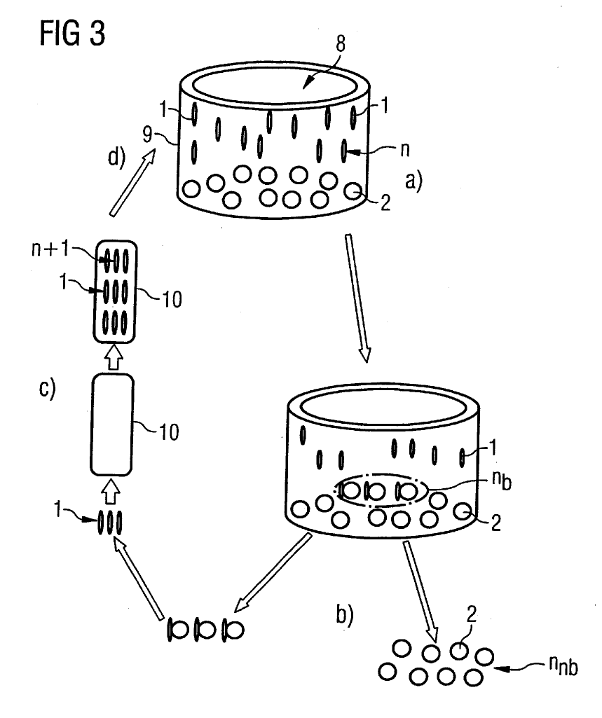

The method to produce a specific phage 1 binding to a specific target molecule proceeds as follows (FIG. 3): In a first step a), a container 9 (for example a beaker, a test tube or a pharmaceutical reactor in which an aqueous solution 8 (for example an isotonic common salt solution) is located is provided. Depending on the later usage conditions of the medicine, the aqueous solution 8 can be modified with regard to its pH value, its temperature, the substances dissolved in it etc. Target molecules 2 are introduced into the aqueous solution 8. These can be dissolved or immobilized on the container walls or on separate carrier structures. Entire cells of the pathological tissue or even entire portions of this can also be used instead of molecules. Furthermore a population n of a virus (advantageously of the M13 phage 1) is introduced into the aqueous solution 8. A contact between the phages 1 and the target molecules 2 can be assisted, possibly by shaking, stirring or the like. The individual virus particles of a virus population are naturally not entirely identical due to spontaneous mutations with regard to their coat proteins. Therefore, a relatively large probability exists that virus particles are located among them that possess at least a certain binding affinity to the target molecules and therefore remain adhered to them. In a method step b), a sub-population n_{b} adhering to the target molecules 2 is separated from the remaining, non-binding population n_{nb}. This can ensue by elution of the aqueous medium 8, for example. In the case of target molecules 2 immobilized in the container, the binding sub-population n_{b} remains in the container. After such a separation (or a separation conducted in another manner) of the binding population from the non-binding population, the binding between the target molecules 2 and the phages 1 of the binding population n_{b} is separated, for instance via addition of an electrolyte or the like. In a subsequent reproduction step, the binding sub-population n_{b} is bred with the aid of bacteria 10. In a last method step the reproduced phage population (n+1) is now introduced again into the aqueous solution 8, wherein the method steps a) through d) are repeated. If necessary the chemical and/or physical and/or physiological properties of the aqueous solution 8 are thereby modified so that tougher conditions increasing the selection pressure exist for the phages, for example. Very generally, the development of the binding coating proteins can be designed such that very specific properties are produced. For instance, it is conceivable that the reaction conditions are selected so that a virus or phage with a very high selectivity (i.e. a biomarker) is obtained that only binds with high sensitivity to a very specific target molecule. Tissues of different clinical pictures can be distinguished with the aid of multiple such biomarkers acting specifically and highly selectively. For instance, prostate cancer can be differentiated from prostatitis. The breeding of the phages 1 or of viruses in general can also ensue with the aid of the described method such that additional binding locations on the phage coat are achieved that, for example, are suitable for tethering of a label 3 of the aforementioned type, for example of a microbubble 5, a ferromagnetic metal particle or a dye absorbing and/or fluorescing in the near infrared range.

Although further modifications and changes may be suggested by those skilled in the art, it is the intention of the inventors to embody within the patent warranted hereon all changes and modifications as reasonably and properly come within the scope of their contribution to the art.

We claim as our invention:

1. A diagnosis substance for application in a method for diagnosis of pathological tissue, said diagnosis substance comprising at least one virus population with virus particles specifically binding to target molecules typical of a specific pathological tissue, and a label detectable with a detection device being respectively bound to the virus particles.

2. A diagnosis substance according to claim 1, comprising a bacteriophage population.

3. A diagnosis substance according to claim 2 wherein the bacteriophage population is the phage M13.

4. A diagnosis substance according to claim 1 wherein said label is detectable by an imaging detection device.

5. A diagnosis substance according to claim 1 wherein said at least one virus population specifically binds to a molecule typical of a cancer tissue.

6. A diagnosis substance according to claim 5, wherein said virus population specifically binds to a target molecule typical of a specific stage of a cancer tissue.

7. A diagnosis substance according to claim 6 wherein said virus population binds to CEACAM-1.

8. A diagnosis substance according to claim 6 wherein said virus population binds to a growth factor of a cancer tissue.

9. A diagnosis substance according to claim 8 wherein said virus population binds to VEGF.

10. A diagnosis substance according to claim 8, wherein said virus population binds to alpha(V)-beta(3)-integrin.

11. A method for production of a diagnosis substance which contains at least one virus population with virus particles specifically binding to target molecules typical of a specific pathological tissue, comprising the steps of:
a) introducing target molecules and a population (n) of a genetically variable virus together into an aqueous solution;
b) separating non-binding viruses from binding viruses;
c) multiplying the binding viruses to obtain a reproduced virus population;
d) introducing the reproduced virus population into the aqueous solution; and
repeating steps a) through d), if necessary with modified reaction conditions, until a virus population is found that specifically binds to a specific target molecule.

12. A method according to claim 11 comprising using bacteriophage population and implementing step c) with the use of bacteria.

13. A method according to claim 12, using the phage M13.

14. A method according to claim 11 comprising using CEACAM-1 molecules as said target molecules.

15. A method according to claim 11 comprising using VEGF molecules as said target molecules.

16. A method according to claim 11 comprising using alpha(V)-beta(3)-integrins as said target molecules.

17. A method according to claim 11 comprising introducing molecules or particles serving as detectable labels into the aqueous solution instead of target molecules.
